(19) United States
(12) Patent Application Publication
(10) Pub. No.: US 2015/0175751 A1
(43) Pub. Date: Jun. 25, 2015

(54) LONG-WEARING TRANSFER RESISTANT FILM-FORMING INGREDIENT

(71) Applicant: Avon Products, Inc., New york, NY (US)

(72) Inventors: Allwyn Colaco, Morristown, NJ (US); Jody P. Ebanks, Nyack, NY (US); Irina Travkina, River Edge, NJ (US)

(21) Appl. No.: 14/409,009

(22) PCT Filed: Mar. 14, 2014
(86) PCT No.: PCT/US14/28420

§ 371 Date: Dec. 18, 2014

Related U.S. Application Data
(60) Provisional application No. 61/789,081, filed on Mar. 15, 2013.

(51) Int. Cl. C08G 77/398; A61K 8/29; A61Q 1/06

(52) U.S. Cl. CPC C08G 77/398; A61Q 1/06; A61K 8/29; A61K 2800/43; A61K 2800/54

(57) ABSTRACT

Cosmetic compositions having improved transfer resistance are provided. The compositions the reaction product of a transition metal, such a titanium ethoxide or tantalum ethoxide, and a hydroxyl-functionalized silicone polymer or resin.

### RELATED APPLICATIONS

This application claims priority benefit to U.S. Provisional Patent Application Ser. No. 61/789,081 filed on Mar. 15, 2013, the entirety of which is herein incorporated by reference for all purposes.

### FIELD OF INVENTION

The invention relates generally to a novel film forming ingredient for imparting substantivity and transfer resistance to a cosmetic film. The film-forming ingredient according to the invention comprises the reaction product of a transition metal alkoxide with a silicone resin having free hydroxyl groups capable of complexing or covalently reacting with the transition metal.

### BACKGROUND OF THE INVENTION

It has long been considered desirable to provide cosmetic products, such as lipsticks, which impart colored films to the skin. Conventionally, color cosmetics comprise pigments dispersed in a base of fats, oils, or waxes. However, a notable disadvantage of traditional color cosmetic products is the propensity to transfer from the lips on contact with napkins, drinking glasses, clothing and the like.

Various polymeric film formers have been added to color cosmetics so as to improve skin substantivity and enhance transfer resistance. Improvements in transfer resistance have been achieved using various silicone resins, including MQ resins. Japanese Patent Application 61-161211 and Japanese Patent Application 61-158913, incorporated by reference herein, both assigned to Shiseido, disclose cosmetic compositions comprising MQ resins having improved resistance to sweat and oil and which do not transfer to objects such as drinking glasses.

There is, however, room for improvement. There is an ongoing need for cosmetic compositions that are long-wearing and transfer resistant. It is therefore an object of the present invention to provide cosmetic compositions, such as lipstick, lip gloss, mascara, and nail enamels, which have reduced propensity to transfer on contact with surfaces such as napkins, clothes, utensils, and drinking glasses.

The foregoing discussion is presented solely to provide a better understanding of nature of the problems confronting the art and should not be construed in any way as an admission as to prior art nor should the citation of any reference herein be construed as an admission that such reference constitutes “prior art” to the instant application.

### SUMMARY OF THE INVENTION

In accordance with the foregoing objectives and others, the present invention provides methods and compositions for forming durable deposits on a human integument, including human hair and skin, and in particular the lips.

In one aspect of the invention, compositions are provided comprising a new film forming composition which is the reaction product of a transition metal alkoxide (e.g., ethoxide) with a hydroxyl-functionalized silicone polymer. The transition metal may be, for example, titanium or tantalum. The transition metal may be a titanium alkoxide having the form Ti(OR)₄ where R is independently selected at each occurrence from lower alkyl radicals (e.g., ethyl). The transition metal may be a tantalum alkoxide having the form Ta(OR)₅ where R is independently selected at each occurrence from lower alkyl radicals (e.g., ethyl). The transition metal alkoxide may, for example, have the form Ti(OCH₂CH₃)₄ or Ta(OCH₂CH₃)₅.

The transition metal alkoxide is reacted with a hydroxyl-functionalized silicone polymer. The hydroxyl-functionalized silicone polymer may be a dimethiconol, a silicone resin, including an MQ resin, having Si—OH content. Without wishing to be bound by any theory, it is believed that the hydroxyl groups form a covalent or coordinate bond with the transition metal, thereby creating a three-dimensional network. The film-forming ingredient also has substantivity to keratinous surfaces, further enhancing the durability and transfer resistance of the silicone polymer or resin.

The weight ratio of transition metal to silicone polymer or resin will typically be from about 5:1 to about 1:1000, or from about 1:1 to about 1:750, or from about 1:10 to about 1:500 or from about 1:25 to about 1:100 (e.g., about 1:50).

In one embodiment of the invention the transition metal is a titanium alkoxide (e.g., Ti(OCH₂CH₃)₄) and the weight ratio of the titanium alkoxide to silicone polymer or resin typically will be from about 5:1 to about 1:1000, or from about 1:1 to about 1:750, or from about 1:10 to about 1:500 or from about 1:25 to about 1:100 (e.g., about 1:50).

In another embodiment the transition metal is a tantalum alkoxide (e.g., Ta(OCH₂CH₃)₅) and the weight ratio of tantalum alkoxide to silicone polymer or resin will typically be from about 5:1 to about 1:1000, or from about 1:1 to about 1:750, or from about 1:10 to about 1:500 or from about 1:25 to about 1:100 (e.g., about 1:50).

In another aspect of the invention, a method is provided for forming an oil transfer resistant film, a water transfer resistant film, a long-wearing film, a comfortable film, and/or a glossy film on the lips comprising applying to the lips a composition according to the invention.

In another aspect of the invention, a method is provided for forming an oil transfer resistant film, a water transfer resistant film, and/or long-wearing film on hair. The film may further include a shine enhancer and provide durable shine to the hair.

In one aspect of the invention, a film forming composition having improved wear and comfort is provided, comprising a reaction product of a transition metal alkoxide (e.g. a tantalum alkoxide or titanium alkoxide) with an MQ resin/dimethiconol crosspolymer. The ratio of the transition metal alkoxide to the MQ resin/dimethiconol crosspolymer typically will be from about 3:1 to about 1:500, or from about 1:1 to about 1:10, or from about 1:1 to about 1:3, or about 1:1.67.

These and other aspects of the present invention will become apparent to those skilled in the art after reading of the following detailed description of the invention, including the appended claims.

### BRIEF DESCRIPTION OF THE DRAWINGS

FIG. 1 shows the results of an experiment assessing the durability of shine of a hair treatment comprising a tantalum/dimethiconol film-former of the invention.

### DETAILED DESCRIPTION

All amounts provided in terms of weight percentage are relative to the entire composition unless otherwise stated. Unless otherwise provided, the term “alkyl” is intended to embrace straight-chained, branched, or cyclic hydrocarbons, particularly those having from one to 20 carbon atoms, and more particularly C₁₋₁₈ or C₁₋₁₆ or C₁₋₁₂ or C₁₋₁₀ or C₁₋₆ hydrocarbons. The term “lower alkyl” includes, without limitation methyl, ethyl, propyl, isopropyl, butyl, n-butyl, iso-butyl, tert-butyl, pentyl, neo-pentyl, amyl, and hexyl, including cyclized analogues thereof. Unless otherwise provided, the term “alkenyl” is intended to embrace straight-chained, branched, or cyclic hydrocarbons, particularly those having from one to 20 carbon atoms, and more particularly C₁₋₁₈ or C₁₋₁₆ or C₁₋₁₂ or C₁₋₁₀ or C₁₋₆ hydrocarbons, which may include one, two, three, or more unsaturated carbon-carbon bonds, which may be in the E or Z configuration.

The compositions of the invention are useful for application to the human integumentary system, including, skin, lips, nails, hair, and other keratinous surfaces. As used herein, the term “keratinous surface” refers to keratin-containing portions of the human integumentary system, which includes, but is not limited to, skin, lips, hair (including eyebrows and eyelashes), and nails (toenails, fingernails, cuticles, etc.) of mammalians, preferably humans. A “keratin fiber” includes hair of the scalp, eyelashes, eyebrows, facial hair, and body hair such as hair of the arms, legs, etc.

The film forming ingredient of the invention is formed by mixing a transition metal compound of the form M(OR)x (where M is a metal and R is, independently each of the x occurrences, a C₁₋₂₂ hydrocarbon, such as ethyl, etc.) with a hydroxyl-containing silicone polymer or resin. The metal may be, without limitation, titanium or tantalum.

The transition metal compound may be, for example, a titanate, such as an alkoxytitanate of the form Ti(OR)₄ where R is independently selected at each occurrence from lower alkyl radicals. In some embodiments, R is the same at each occurrence. In some embodiments R may be different at least two occurrences (e.g., a mixed alkoxy titanate). R may be, without limitation, a C₁₋₂₂ or C₁₋₁₈ or C₁₋₁₆ or C₁₋₁₂ or C₁₋₁₀ or C₁₋₆ or C₁₋₄ hydrocarbon. In one embodiment, R is lower alkyl, including without limitation, methyl, ethyl, propyl, isopropyl, n-butyl, tert-butyl, isobutyl, pentyl, neo-pentyl, amyl, or hexyl. R may comprise an unsaturated bond, such as vinyl, allyl, ethyleneyl, propenyl, hexenyl, or the like. R may also comprise an aromatic group, for example, phenyl or benzyl.

In one embodiment of the alkoxytitanate of the form Ti(OR)₄, at least one of R is methyl. In another embodiment, at least two of R are methyl. In another embodiment, at least three of R are methyl. In another embodiment, all four occurrences of R are methyl. In another embodiment of the alkoxytitanate of the form Ti(OR)₄, at least one of R is ethyl. In another embodiment, at least two of R are ethyl. In another embodiment, at least three of R are ethyl. In another embodiment, all four occurrences of R are ethyl. A preferred titanium alkoxide is Ti(OCH₂CH₃)₄.

The transition metal compound also may be, for example, a tantalum compound, such as an alkoxytantalate of the form Ta(OR)₅ where R is independently selected at each occurrence from lower alkyl radicals. In some embodiments, R is the same at each occurrence. In some embodiments R may be different at least two occurrences (e.g., a mixed alkoxy titanate). R may be, without limitation, a C₁₋₂₂ or C₁₋₁₈ or C₁₋₁₆ or C₁₋₁₂ or C₁₋₁₀ or C₁₋₆ or C₁₋₄ hydrocarbon. In one embodiment, R is lower alkyl, including without limitation, methyl, ethyl, propyl, isopropyl, n-butyl, tert-butyl, isobutyl, pentyl, neo-pentyl, amyl, or hexyl. R may comprise an unsaturated bond, such as vinyl, allyl, ethyleneyl, propenyl, hexenyl, or the like. R may also comprise an aromatic group, for example, phenyl or benzyl.

In one embodiment of the alkoxytantalate of the form Ta(OR)₅, at least one of R is methyl. In another embodiment, at least two of R are methyl. In another embodiment, at least three of R are methyl. In another embodiment, at least four of R are methyl. In another embodiment, all five occurrences of R are methyl. In another embodiment of the alkoxytantalate of the form Ta(OR)₅, at least one of R is ethyl. In another embodiment, at least two of R are ethyl. In another embodiment, at least three of R are ethyl. In another embodiment, at least four of R are ethyl. In another embodiment, all five occurrences of R are ethyl. A preferred alkoxytantalate is Ta(OCH₂CH₃)₅.

In one embodiment, the hydroxyl-functionalized silicone polymer may have the structure:

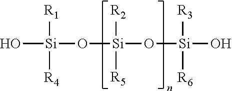

Where R₁-R₆ is independently a C₁₋₁₀ or C₁₋₆ or C₁₋₄ hydrocarbon (aliphatic or aromatic) substituent. R₁-R₆ may be selected from methyl, ethyl, propyl, isopropyl, butyl, isobutyl, tert-butyl, pentyl, hexyl, vinyl, allyl, ethyleneyl, propenyl, hexenyl, phenyl, benzyl, cyclopentyl, and cyclohexyl, as well as halogenated (fluoro, chloro, etc.) and perhalogenated (e.g., perfluorinated) analogs thereof, and wherein each of R₁-R₆ may further include 1-4 (e.g., one, two, three, or four) substituents selected from hydroxyl, alkoxyl, polyalkyleneoxide (e.g., polyethylene oxide), amino, alkyl amino, and dialkylamino Integer “n” may range from 2 up to 200, 500, 800, 1,000, 1,500, 2,000 or more, such that the viscosity is, for example, from 20 to 1,000,000 cps or greater (or from 50 to 500,000 cps or from 100 to 5,000 cps) at 25° C. In one embodiment at least one of R₂ and R₅ is methyl. In one embodiment R₂ and R₅ are both methyl. In one embodiment R₁-R₆ are all methyl. In one embodiment, the silicone polymer is dimethiconol.

In one embodiment, the hydroxyl-functionalized silicone polymer is an MQ resin, where “M” refers to units of the form R₃SiO_{1/2} and “Q” refers to units of the form SiO_{4/2}. The MQ resins of the invention may further have some content (typically minor, or up to 5 mole %) of “D” units of the form R₂SiO and “T” units of the form RSiO_{3/2}, as well as other silicon-containing units of the form RₙSiXₘO_{y}, where R is a C₁₋₁₀ or C₁₋₆ or C₁₋₄ hydrocarbon (aliphatic or aromatic) substituent (such as methyl, ethyl, propyl, butyl, pentyl, hexyl, vinyl, allyl, ethyleneyl, propenyl, hexenyl, phenyl, cyclopentyl, cyclohexyl, etc.), and X is a functional group H, OH, Cl or OR. In some embodiments, R is methyl. In some embodiments, R is methyl at 90% or more the occurrences. In some embodiments, R is methyl at 95% or more the occurrences. The ratio of M to Q units may be, for example, about 5:1 to about 1:5 or about 2:1 to about 1:2 or about 3:2 to about 2:3. MQ resins may have the INCI name Trimethylsiloxysilicate. Suitable MQ resins include those that have some residual hydroxyl content, as the hydroxyl functional group is necessary for complexing with or bonding to titanium and/or tantalum. The hydroxyl group may be bonded to silicon (i.e., silanol). The silanol content of the MQ resin may range from about 0.1% to about 10% by weight, or from about 0.2% to about 5% by weight, or from about 1% to about 3% by weight of the MQ resin. The hydroxyl group may be in the form of a dimethylhydroxysiloxy unit of the form (HO)(CH₃)₂SiO_{1/2}.

One suitable MQ resin is BELSIL® TMS 803 from Wacker which is the co-hydrolysis product of tetraalkoxysilane (Q unit) and trimethylethoxysilane (M unit). The chemical structure of this MQ resin can be seen as a three-dimensional network of polysilic acid units, which are endblocked with trimethylsilyl groups and retain some residual ethoxy and hydroxy functional groups.

In another embodiment, the MQ resin may be fluorinated. US Patent Pub. 2006/0229424, the disclosure of which is hereby incorporated by reference, describes reacting MQ resins with fluoroalkylsilanes. In another embodiment, the MQ resin may be a MQ/Propyl silsesquioxane (T propyl) resin, as disclosed in, for example, U.S. Pat. No. 7,803,358, WO2005075542 and WO200507556, the disclosures of which are hereby incorporated by reference. A number of derivatives of MQ resins are contemplated to be useful in the practice of the invention, the importance being that the resin comprises sufficient hydroxyl functional groups for complexing or reacting with the transition metal (e.g., titanium or tantalum).

In another embodiment, the hydroxyl-containing silicone polymer is a Trimethylsiloxysilicate/Dimethiconol Crosspolymer (INCI). Such material may comprise a crosspolymer of an MQ resin with Dimethiconol. This polymer provides additional hydroxyl groups at the end-caps which are capable of reacting with the transition metal (e.g., titanium or tantalum). One example is Dow Corning® 7-4405 Cosmetic Fluid.

The novel film-former of the invention is formed by mixing the hydroxyl-functionalized silicone polymer with titanium alkoxide or tantalum alkoxide, optionally in a suitable solvent, such as isododecane (IDD) or cyclomethicone. The reaction is exothermic.

The titanium/silicone or tantalum/silicone film-former of the invention may be added to any cosmetic where a transfer resistant, durable, and substantive film is desired.

The cosmetic compositions of the invention may comprise any conventional components, including pigments and colorants, fillers and cosmetic powders, additional film formers, gelling agents, thickeners, conditioners, actives, solvents, emulsifiers, humectants, emollients, pH adjusters, antioxidants, preservatives, fragrances, and the like.

For example, the compositions may, for example, comprise (1) from about 1% to about 75% (e.g., about 1-10%, about 10-20%, about 30-40%, about 40-50%, or about 50-60%) by weight of an oil, including without limitation hydrocarbons (e.g., isododecane), fatty alcohols, ester oils, and silicone oils; (2) from about 0.5% to about 20% (e.g., about 0.5-2.5%, or about 2.5%-5%, or about 5-10%, or about 10-15%, or about 15-20%) by weight of a film-forming polymer, including a polyurethane film former, such as bis-PEG-1 dimethicone-propylene glycol/IPDI copolymer; (3) from about 1% to about 25% (e.g., about 1%-5%, or about 5-10%, or about 10-15%, or about 15-20%) by weight of a gelling agent, such as Isododecane (and) Disteardimonium Hectorite (and) Propylene Carbonate (INCI); (4) from about 0.1% to about 15% (e.g., about 0.1-1%, or about 1-2.5%, or about 2.5-5%, or about 5-10%, or about 10-15%) by weight of a gelling silicone such as Dimethicone/Vinyl Dimethicone Crosspolymer; (5) from about 1% to about 35% (e.g., about 8% to about 20%) by weight pigments; and/or (6) from about 0.1% to about 2.5% (e.g., about 0.1-0.5%, about 0.5-1%, or about 1-2.5%) by weight caprylyl glycol.

The composition may also comprise colorants such as dyes, pigments and lakes. As used herein, the term “pigments” embraces lakes and fillers such as talc, calcium carbonate, etc. Exemplary inorganic pigments include, but are not limited to, inorganic oxides and hydroxides such as magnesium oxide, magnesium hydroxide, calcium oxide, calcium hydroxides, aluminum oxide, aluminum hydroxide, iron oxides (α-Fe₂O₃, γ-Fe₂O₃, Fe₃O₄, FeO) and iron hydroxides including red iron oxide, yellow iron oxide and black iron oxide, titanium dioxide, titanium lower oxides, zirconium oxides, chromium oxides, chromium hydroxides, manganese oxides, manganese hydroxides, cobalt oxides, cobalt hydroxides, cerium oxides, cerium hydroxides, nickel oxides, nickel hydroxides, zinc oxides and zinc hydroxides and composite oxides and composite hydroxides such as iron titanate, cobalt titanate and cobalt aluminate and the like. Preferably, the inorganic oxide particles may be selected from silica, alumina, zinc oxide, iron oxide and titanium dioxide particles, and mixtures thereof. In one embodiment, the pigments have a particle size from 5 nm to 500 microns, or from 5 nm to 250 microns, or from 10 nm to 100 microns. In some embodiments, the particle size (median) will be less than bout 5 microns or less than 1 micron.

Additional exemplary color additive lakes include, for example: D&C Red No. 19 (e.g., CI 45170, CI 73360 or CI 45430); D&C Red No. 9 (CI 15585); D&C Red No. 21 (CI 45380); D&C Orange No. 4 (CI 15510); D&C Orange No. 5 (CI 45370); D&C Red No. 27 (CI 45410); D&C Red No. 13 (CI 15630); D&C Red No. 7 (CI 15850:1); D&C Red No. 6 (CI 15850:2); D&C Yellow No. 5 (CI 19140); D&C Red No. 36 (CI 12085); D&C Orange No. 10 (CI 45475); D&C Yellow No. 19 (CI 15985); FD&C Red #40 (CI#16035); FD&C Blue #1 (CI#42090); FD&C Yellow #5 (CI#19140); or any combinations thereof.

Suitable fillers may include talc, silica, alumina, zinc stearate, mica, kaolin, nylon (in particular orgasol) powder, polyethylene powder, polypropylene powder, acrylates powders, Teflon, starch, boron nitride, copolymer microspheres such as Expancel (Nobel Industrie), Polytrap (Dow Corning), and silicone resin microbeads (Tospearl from Toshiba).

Other fillers that may be used in the compositions of the invention include inorganic powders such as chalk, fumed silica, fumed alumina, calcium oxide, calcium carbonate, magnesium oxide, magnesium carbonate, Fuller's earth, attapulgite, bentonite, muscovite, phlogopite, synthetic mica, lepidolite, hectorite, biotite, lithia mica, vermiculite, aluminum silicate, aluminum magnesium silicate, diatomaceous earth, starch, alkyl and/or trialkyl aryl ammonium smectites, chemically modified magnesium aluminum silicate, organically modified montmorillonite clay, hydrated aluminum silicate, hydrated silica, fumed aluminum starch octenyl succinate barium silicate, calcium silicate, magnesium silicate, strontium silicate, metal tungstate, magnesium, silica alumina, zeolite, barium sulfate, calcined calcium sulfate (calcined gypsum), calcium phosphate, fluorine apatite, hydroxyapatite, ceramic powder, metallic soap (zinc stearate, magnesium stearate, zinc myristate, calcium palmitate, and aluminum stearate), colloidal silicon dioxide; organic powder, cyclodextrin, methyl polymethacrylate powder, copolymer powder of styrene and acrylic acid, benzoguanamine resin powder, and poly(ethylene tetrafluoride) powder.

The compositions may include natural or synthetic film-forming polymers. Suitable polymeric film formers include polyolefins, silicone polymers (e.g., dimethicones, dimethiconols, amodimethicones, silicone resins, etc.), (meth)acrylates, alkyl(meth)acrylates, polyurethanes, fluoropolymers, silicone polyurethanes, and silicone acrylates such as acrylates/dimethicone copolymers. In some embodiments, it may be desirable to add a hydrophilic or water-soluble film former (e.g., cellulosics, polysaccharides, polyquaterniums (such as polyquaternium-37 (INCI), etc.) to the composition to improve spreading, emulsion stability, aesthetic look and feel, etc. Elastomers formed from ethylene, propylene, butylene, and/or styrene monomers may also be useful.

The compositions of the invention will typically include a cosmetically or dermatologically acceptable vehicle, which may be in the form of, for example, a serum, a cream, a lotion, a gel, or a stick, and may comprise an emulsion (e.g., water-in-oil, oil-in-water, water-in-silicone, silicone-in-water, polyol-in-silicone, silicone-in-polyol emulsion, etc.), or may comprise an aqueous or ethanolic vehicle, silicone (e.g., cyclomethicone, dimethicone, etc.), hydrocarbon (e.g., petrolatum, isododecane, etc.), ester oil (isopropyl myristate, myristyl myristate, or the like). The vehicle may be anhydrous and may comprise oils, such as dimethicones, hydrocarbons (e.g., isododecane), petrolatum, ester oils, and the like. The vehicle may further comprise an emulsifier, gelling agent, structuring agent, rheology modifier (e.g., a thickener), film former, or the like. The vehicle may comprise from about 25% to about 99% by weight of the composition.

The compositions may further include an emulsifier. The amount of emulsifier will typically be from about 0.001 to about 10% by weight, but preferably will range from about 0.01 to about 5% by weight, and most preferably about 0.1 to about 1% by weight, based upon the total weight of the composition. The emulsifier may be ionic, zwitterionic, or nonionic. Suitable emulsifiers include those of the polyethoxylated type (e.g., polyoxyethylene ethers or esters), polydiorganosiloxane-polyoxyalkylene block copolymers (e.g., dimethicone copolyol), Steareth-20, Steareth-21, fatty alcohols (e.g., Cetearyl Alcohol), Polyoxethylene sorbitan fatty acid esters (i.e., polysorbates), and Hydrogenated Castor Oil, to name a few. Additional emulsifiers are provided in the NCI Ingredient Dictionary and Handbook 11th Edition 2006, the disclosure of which is hereby incorporated by reference.

The composition may further include a gelling agent. The gelling agent may comprise, for example, a silicone resin, including Dimethicone/Vinyl Dimethicone crosspolymer, silicone T-resins, ETPEA, polyamides, cellulose ethers (e.g., methyl cellulose or ethyl cellulose) and the like. Thickeners such as acrylates copolymers, hydroxyalkyl cellulose, carboxymethylcellulose, carbomers, and vegetable gums such as xanthan gum may be included.

The composition may also comprise humectants such as polyols (e.g., glycols), including without limitation, glycerin, propylene glycol, ethoxydiglycol, butylene glycol, pentylene glycol, hexylene glycol, caprylyl glycol, and the like. These will typically be added in amount from about 0.001 to about 5% by weight.

In another embodiment, the topical compositions of the present invention may also include one or more of the following: a skin penetration enhancer; an emollient, such as isopropyl myristate, petrolatum, volatile or non-volatile silicones oils (e.g., methicone, dimethicone), ester oils, mineral oils, and fatty acid esters; a humectant, such as glycerin, hexylene glycol or caprylyl glycol; a skin plumper, such as palmitoyl oligopeptide, collagen, collagen and/or glycosaminoglycan (GAG) enhancing agents; an exfoliating agent; and an antioxidant (e.g., TDPA).

The compositions of the invention may optionally include additional skin benefit agents such as emollients (dimethicone oils, ester oils, or hydrocarbon oils), humectants (e.g., polyols, including propylene glycol, glycerin, etc.), antioxidants (e.g., BHT, ascorbic acid, sodium ascorbate, ascorbyl palmitate, beta-carotene, etc.), vitamins (e.g., tocopherol, tocopheryl acetate, etc.), alpha-hydroxy acids (e.g., glycolic acid), beta-hydroxy acids (e.g., salicylic acid), retinoids (e.g., retinoic acid, all-trans-retinoic acid, retinaldehyde, retinol, and retinyl esters such as acetates or palmitates), other anti-aging ingredients (e.g., collagen stimulators), and depigmenting agents (e.g., TDPA).

A sunscreen may be included to protect the skin from damaging ultraviolet rays. In an illustrative embodiment of the present disclosure, the sunscreen provides both UVA and UVB protection, by using either a single sunscreen or a combination of sunscreens. Among the sunscreens that can be employed in the present compositions are avobenzone, cinnamic acid derivatives (such as octylmethoxy cinnamate), octyl salicylate, oxybenzone, octocrylene, titanium dioxide, zinc oxide, or any mixtures thereof. The sunscreen may be present from about 1 wt % to about 30 wt % of the total weight of the composition.

The compositions may comprise a cationic polymer. Cationic polymers include, but are not limited to, polyquaternium 4, polyquaternium 6, polyquaternium 7, polyquaternium 10, polyquaternium 11, polyquaternium 16, polyquaternium 22, polyquaternium 28, polyquaternium 32, and guar hydroxypropyltrimonium chloride. When present, the cationic polymer will typically comprise an amount of about 0.1% to about 15% (w/w) of the composition. In other embodiments the compositions may contain an amount of cationic (quaternium) ingredients that are anhydrous or have very low level of water, e.g., less than 1% by weight. Other suitable quaternium compounds include, without limitation, Polyquaternium-37 (INCI), Silicone Quaternium-18 (INCI), PEG-2 Dimeadowfoamamidoethylmonium Methosulfate and Hexylene Glycol (INCI), and Cetrimonium Chloride (INCI), to name a few. Such quaternium compounds, if present, will typically comprise from about 0.05% to about 5% by weight of the total composition, and more typically, from about 0.1% to about 1.5% by weight.

The compositions may also comprise monomer quaternary ammonium compounds such as, for example, alkyltrimethylammonium chlorides, dialkylmethyl-ammonium chlorides, alkyldimethylbenzylammonium chlorides, and alkylpyridinium chlorides. In one embodiment, the composition comprises at least one hair conditioning agent selected from the group consisting of polyquaterniums, cationic polymers, cationic surfactants, non-volatile dimethicone oils, dimethiconols, amodimethicones, ester oils, fatty alcohols, cationic gums and cellulosics, amidoamines, cetrimonium chloride, behentrimonium chloride, stearamidopropyl dimethylamine, polyesteramines, and cationically charge-modified polymers derived from guar gum, cellulose, proteins, polypeptides, chitosan, lanolin, starches and amino silicones.

The compositions may include a nonionic surfactant such as Laureth-23, Ceteth-10, Ceteth-20, IsoCeteth-20, Steareth-20, Oleth-10, Oleth-20, or alkyl polyglucose. The nonionic surfactant may be formed from a fatty alcohol, a fatty acid, or a glyceride with a C8 to C24 carbon chain. The compositions of the invention can further comprise proteins, peptides, and amino acids including hydrolyzed soy protein, lauryldimonium hydrolyzed soy protein (cationic Soya protein), wheat amino acids, corn, wheat, milk, or silk proteins, collagens, keratins, taurine and arginine hydrochloride, etc.

The cosmetic compositions of the invention may optionally include one or more agents that provide or enhance shine. Shine enhancing agents will typically have a refractive index greater than about 1.4, preferably greater than about 1.5 when measured as a film at 25° C. Suitable shine enhancing agents include without limitation, polyols, fatty esters, silicone phenylpropyldimethylsiloxysilicate, polybutene, polyisobutene, hydrogenated polyisobutene, hydrogenated polycyclopentadiene, propyl phenyl silsesquioxane resins; lauryl methicone copolyol, perfluorononyl dimethicone, dimethicone/trisiloxane, methyl trimethicone, and combinations thereof. In one embodiment, the composition will comprise a shine-enhancing agent in an amount from about 0.1% to about 10% by weight, based on the total weight of the composition.

Other suitable components include those agents that provide a prophylactic or therapeutic benefit to skin. Particular mention may be made of alpha-hydroxy acids, beta hydroxyl acids, ascorbic acid or Vitamin C and derivatives thereof (e.g., C₁-C₈ esters thereof); retinoids such as retinol (Vitamin A) and the esters thereof (e.g., C₁-C₈ esters, such as palmitate), retinoic acid and the derivatives thereof, hyaluronic acid, chemical sun screens useful in the cosmetic field including any UVA and UVB filter useful in the cosmetic field including mixtures thereof and blends with physical filters including, but not limited to metal oxide particles such as titanium oxides and/or zinc oxides. Additional benefit agents include botanicals, thiodipropionic acid (TDPA) and esters thereof; (e.g., retinoic acid, all-trans-retinoic acid, retinaldehyde, retinol, and retinyl esters such as acetates or palmitates, and others); alpha-hydroxy acids (e.g., glycolic acid), beta-hydroxy acids (e.g., salicylic acid and salicylates); exfoliating agents (e.g., glycolic acid, 3,6,9-trioxaundecanedioic acid, etc.), depigmenting agents (e.g., hydroquinone, kojic acid, etc.) estrogen synthetase stimulating compounds (e.g., caffeine and derivatives); compounds capable of inhibiting 5 alpha-reductase activity (e.g., linolenic acid, linoleic acid, finasteride, and mixtures thereof); antioxidants (e.g., BHT, ascorbic acid, sodium ascorbate, ascorbyl palmitate, beta-carotene, thiodipropionic acid, vitamin E, etc.), barrier function enhancing agents (e.g., ceramides, glycerides, cholesterol and its esters, alpha-hydroxy and omega-hydroxy fatty acids and esters thereof, etc.); collagenase inhibitors; and elastase inhibitors; to name a few. These benefit agents will typically be present, if at all, in amounts between about 0.001% and about 10% by weight of the composition.

Other additives include: vitamins, such as tocopherol and ascorbic acid; vitamin derivatives such as ascorbyl monopalmitate, tocopheryl acetate, and Vitamin E palmitate; metal chelating agents such as EDTA or salts thereof; and pH adjusters (citric acid, ethanolamine, sodium hydroxide, etc.).

The compositions typically comprises a preservative or anti-microbial agent, for example, methylchloroisothiazolinone, methylisothiazolinone, methylparaben, propylparaben, phenoxyethanol, or caprylyl glycol.

The composition can be applied as often as necessary to impart the desired cosmetic finish, color or appearance to the lip, nails, hair and/or skin. A composition according to the invention is expected to achieve transfer resistance, comfort, and optionally gloss, and remain on the integument (hair, skin, lips, nails, etc.) for a long-wear period such as from about 1 to about 24 hours. The composition is also expected to maintain transfer resistance for a period such as from about 1 hour to about 24 hours. Typically, a long-wear period and/or transfer resistance period may be from about 2-24 hours, or about 4-24 hours, or 8-24 hours.

A variety of evaluation methods of transfer resistance and long wearing properties are known in the cosmetic arts, such as in dry blot, oil blot, and rub tests. For example, U.S. Pat. No. 6,071,503 discloses various methods of evaluating cosmetic properties, the disclosure of which is hereby incorporated by reference.

In one embodiment, the composition is intended for use as a non-therapeutic treatment. In another embodiment, the composition is an article intended to be rubbed, poured, sprinkled, or sprayed on, introduced into, or otherwise applied to the human body for cleansing, beautifying, promoting attractiveness, or altering the appearance, in accordance with the US FD&C Act, §201(i).

### EXAMPLES

### Example 1

Two lip color products were prepared according to the formulas in Table 1. The MQ resin used was Belsil TMS from Wacker. The tantalum ethoxide was Aculon ALA-50. The MQ Resin+Tantalum ethoxide was prepared by mixing 56.6% Belsil TMS (70% resin in IDD) with 1% Aculon ALA-50 in 42.4% IDD. The tantalum ethoxide had a final solids content of 0.063% in the “Invention” formulation. Both compositions “Control A” and “Invention A” comprised 5% by weight of the MQ resin. The IDD added to the “Invention A” formulation was adjusted to account for the extra IDD in the MQ Resin+Tantalum ethoxide preparation. The compositions were prepared by heating Part A to 80-85° C. and milling the material until homogenous. Part B and Part C were then added in sequence, the composition being milled until homogenous after each addition. The Part D ingredients were then added.

TABLE 1

| | Control A | Invention A |
|---|---|---|
| Ingredient | Weight (g) | Weight (g) |
| Part A | | |
| Isododecane (IDD) | 17.78 | 15.60 |
| Bis-PEG-1 dimethicone-propylene | 4.00 | 4.00 g |
| glycol/IPDI copolymer | | |
| MQ Resin (70% resin in IDD) | 2.86 | — |
| MQ Resin + Tantalum ethoxide (in IDD) | — | 5.04 |
| Bentone Gel | 6.00 | 6.00 |
| Caprylyl Glycol | 0.20 | 0.20 |
| Part B | | |
| Pigment Grind | 4.00 | 4.00 |
| Part C | | |
| Dimethicone/Vinyl Dimethicone | 3.00 | 3.00 |
| Part D | | |
| Effect Pigments | 2.16 | 2.16 |

To assess the transfer resistance of each formulation of Table 1, 1 mL drawdowns of each formulation were applied as a film on Vitro skin and allowed to dry for one hour. Droplets of water or artificial sebum were equilibrated on each sample for 15 minutes and then excess liquid was removed. Styrofoam discs attached to a 1 kg weight were rotated once on the sample and the amount of colorant transferred to the Styrofoam disc was evaluated visually. The “Control A” formulation achieved excellent oil resistance but transferred moderately when challenged with water. The addition of 0.126% Aculon ALA-50 (solids of 0.063%) in the “Invention A” formulation markedly improved the water transfer resistance.

This example was repeated with the addition of 16.9% of the MQ resin and 1.1% Aculon ALA-50, and was also repeated with the addition of 16% of the MQ resin and 0.42% Aculon ALA-50. In each case the tantalum ethoxide dramatically improved the water transfer resistance.

### Example 2

Two lip color products were prepared according to the formulas in Table 2. The resin used was Dow Corning® 7-4405 Cosmetic Fluid (DC 7-4405), which is an MQ resin/dimethiconol crosspolymer. The tantalum ethoxide was Aculon ALA-50.

The DC 7-4405 and Aculon ALA 50 were prepared by premixing. The tantalum ethoxide had a final solids content of 1.1%. The compositions were prepared as in Example 1. Control B contained 16.8% resin on a solids basis. Invention B contained 16% resin on a solids basis.

TABLE 2

| | Control B | Invention B |
|---|---|---|
| Ingredient | Weight (g) | Weight (g) |
| Part A | | |
| Isododecane (IDD) | 16.93 | 16.93 |
| Bis-PEG-1 dimethicone-propylene | 10.00 | 10.00 |
| glycol/IPDI copolymer | | |
| MQ Resin | 42.20 | — |
| MQ Resin + Tantalum ethoxide (in IDD) | — | 42.20 |
| Bentone Gel | 10.00 | 10.00 |
| Caprylyl Glycol | 0.50 | 0.50 |
| Part B | | |
| Pigment Grind | 10.00 | 10.00 |
| Part C | | |
| Dimethicone/Vinyl Dimethicone | 5.00 | 5.00 |
| Part D | | |
| Effect Pigments | 5.37 | 5.37 |

To assess the transfer resistance of the formulation in Table 2, 1 mL drawdowns of each formulation were applied as a film on Vitro skin and allowed to dry for one hour. Droplets of water or artificial sebum were equilibrated on each sample for 15 minutes and then excess liquid was removed. Styrofoam discs attached to a 1 kg weight were rotated once on the sample and the amount of colorant transferred to the Styrofoam disc was evaluated visually. The results are presented in Tables 4 and 5 below. The grading scale is presented in Table 3 below.

TABLE 3

| Grading Scale: Criteria | Scale |
|---|---|
| Less than negative control | 1 |
| Equal to or slightly better than negative control | 2 |
| Between negative and positive control | 3 |
| About equal to positive control | 4 |
| Better than positive control | 5 |

TABLE 4
Transfer Test: Control B

| | |
|---|---|
| Water | Oil |

| Substrate | Styrofoam | Substrate | Styrofoam |
|---|---|---|---|
| 4 | 3 | 5 | 5 |
| 4 | 3 | 5 | 5 |
| 4 | 3 | 5 | 5 |
| 4 | 3 | 5 | 5 |
| 4 | 3 | 5 | 5 |

TABLE 5
Transfer Test: Invention B

| | |
|---|---|
| Water | Oil |

| Substrate | Styrofoam | Substrate | Styrofoam |
|---|---|---|---|
| 4 | 5 | 5 | 5 |
| 4 | 4 | 5 | 5 |
| 4 | 4 | 5 | 5 |
| 4 | 5 | 5 | 5 |
| 4 | 5 | 5 | 5 |

This example shows that modification of the MQ/dimethiconol crosspolymer resin with tantalum improves the water transfer resistance of the composition without sacrificing the oil transfer resistance.

### Example 3

Three lip color products were prepared according to the formulas in Table 6. The resin used was Belsil TMS, which is an MQ resin. Titanium ethoxide was used in place of tantalum ethoxide. The first MQ Resin+Titanium ethoxide was prepared by prereacting the Belsil with the titanium ethoxide in IDD. As seen below, this prereaction mixture was utilized as 12.6% of the final Invention C mixture, yielding effective amounts of 5% MQ resin and 0.05% titanium ethoxide in the Invention C formulation. The second MQ Resin+Titanium ethoxide was prepared by prereacting the Belsil with the titanium ethoxide in IDD. As seen below, this prereaction mixture was utilized as 12.6% of the final Invention D mixture, yielding effective amounts of 5% Belsil and 0.1% titanium ethoxide in the Invention D formulation. The total amount of IDD was adjusted to account for the additional IDD used to prepare the MQ resin/titanate material. The compositions were prepared as in Example 1.

TABLE 6

| | Control C | Invention C | Invention D |
|---|---|---|---|
| Ingredient | Weight (g) | Weight (g) | Weight (g) |
| Part A | | | |
| Isododecane (IDD) | 44.46 | 39.00 | 39.00 |
| Bis-PEG-1 dimethicone- | 10.00 | 10.00 | 10.00 |
| propylene glycol/IPDI | | | |
| copolymer | | | |
| MQ Resin (70% resin in IDD) | 7.14 | — | — |
| MQ Resin + Titanium ethoxide | — | 12.60 | 12.60 |
| (in IDD) | | | |
| Bentone Gel | 15.00 | 15.00 | 15.00 |
| Caprylyl Glycol | 0.500 | 0.500 | 0.500 |
| Part B | | | |
| Pigment Grind | 10.00 | 10.00 | 10.00 |
| Part C | | | |
| Dimethicone/Vinyl Dimethicone | 7.50 | 7.50 | 7.50 |
| Part D | | | |
| Effect Pigments | 5.4 | 5.4 | 5.4 |

The transfer resistance of each formulations of Table 6 was determined according to Example 1. The results are presented in Tables 7, 8, and 9 below.

TABLE 7
Transfer Test: Control C

| | |
|---|---|
| Water | Oil |

| Substrate | Styrofoam | Substrate | Styrofoam |
|---|---|---|---|
| 2 | 2 | 5 | 4 |
| 5 | 4 | 5 | 4 |
| 2 | 2 | 5 | 4 |
| 2 | 2 | 5 | 4 |
| 2 | 2 | 5 | 4 |

TABLE 8
Transfer Test: Invention C

| | |
|---|---|
| Water | Oil |

| Substrate | Styrofoam | Substrate | Styrofoam |
|---|---|---|---|
| 4 | 3 | 5 | 4 |
| 4 | 2 | 5 | 4 |
| 4 | 3 | 5 | 4 |
| 4 | 3 | 5 | 4 |
| 4 | 3 | 5 | 4 |

TABLE 9
Transfer Test: Invention D

| | |
|---|---|
| Water | Oil |

| Substrate | Styrofoam | Substrate | Styrofoam |
|---|---|---|---|
| 5 | 4 | 5 | 4 |
| 5 | 4 | 5 | 4 |
| 4 | 3 | 5 | 4 |
| 4 | 2 | 5 | 4 |
| 4 | 3 | 5 | 4 |

This example shows that even addition of very low levels (˜0.1%) of the transition metal improves the water-transfer resistance of the lip gloss.

### Example 4

Three lip color products were prepared according to the formulas in Table 11. Each formulation contained the same lip stick base (“Base”), shown in Table 10. The Part A components were combined in a beaker and then the Part E components were added, and the batch was heated to about 95° C. and mixed until uniform. The mixture was allowed to cool to about 85° C., and then the Part F ingredients were added. The batch was mixed until uniform to yield the base.

TABLE 10

| Ingredient | Weight (g) |
|---|---|
| Part A | |
| Polyglyceryl-2 Diisostearate/IDPI | 3.90 |
| Copolymer | |
| Stearyl/PPG-3 Myristyl Ether Dimer | 4.00 |
| Dilinoleate | |
| Phenyl Trimethicone | 1.65 |
| Hydrogenated | 2.50 |
| Polyisobutene/Polybutene | |
| Lanolin | 3.00 |
| Di-PPG-3 Myristyl Ether Adipate | 2.00 |
| Tegosoft OER | 1.50 |
| Ethyl Macadamiate/Toc./Malic Acid | 1.00 |
| Part E | |
| Polyethylene Wax | 7.00 |
| Ozokerite Wax | 4.00 |
| Microcrystalline Wax | 1.00 |
| Part F | |
| Ethylhexyl-Methoxycinnamate | 7.50 |
| Base Total | 39.05 |

Invention E was prepared with Belsil TMS, an MQ resin from Wacker. Invention F was prepared with Dow Corning® 7-4405 Cosmetic Fluid, an MQ resin/dimethiconol crosspolymer. The titanium ethoxide was obtained from Aculon. Control D was prepared with conventional transfer resistant film formers with no MQ resin and titanium ethoxide. The compositions were prepared by combining all ingredients over heat.

TABLE 11

| Ingredient | Control D | Invention E | Invention F |
|---|---|---|---|
| Base from Table 10 | 39.05 | 39.05 | 39.05 |
| Methyl Trimethicone | 14.50 | 14.50 | 14.50 |
| Polyglyceryl-2-Diisostearate/ | 4.80 | — | — |
| IDPI Copolymer | | | |
| Polyvinyl pyrrolidone/ | 1.20 | — | — |
| Hexadecene copolymer | | | |
| Isododecane (IDD) | 9.00 | — | — |
| MQ Resin (Wacker)¹ + Titanium | — | 15.00 | — |
| ethoxide | | | |
| MQ Resin (Dow)² + Titanium | — | — | 15.00 |
| ethoxide | | | |
| Polyisobutene/Isododecane - | 1.50 | 1.50 | 1.50 |
| blend | | | |
| Castor Oil | 9.95 | 9.95 | 9.95 |
| Pigment Grind | 20.00 | 20.00 | 20.00 |
| Total | 100.00 | 100.00 | 100.00 |

¹Belsil TMS, an MQ resin from Wacker
²Dow Coming ® 7-4405 Cosmetic Fluid (DC 7 4405), an MQ resin/dimethiconol crosspolymer from Dow Corning

The transfer resistance of each formulations of Table 11 was determined according to the protocol of Example 1. The grading scale was 1-5, 1 being the most transfer and 5 being the least transfer. The results are presented in Tables 12, 13, and 14 below.

TABLE 12
Transfer Test: Control D

| | |
|---|---|
| Water | Oil |

| Substrate | Styrofoam | Substrate | Styrofoam |
|---|---|---|---|
| 2 | 3 | 1 | 1 |
| 2 | 3 | 1 | 1 |
| 1 | 3 | 1 | 1 |
| 2 | 3 | 1 | 1 |

TABLE 13
Transfer Test: Invention E

| | |
|---|---|
| Water | Oil |

| Substrate | Styrofoam | Substrate | Styrofoam |
|---|---|---|---|
| 3 | 1 | 1 | 1 |
| 3 | 1 | 1 | 1 |
| 3 | 1 | 1 | 1 |
| 3 | 1 | 1 | 1 |

TABLE 14
Transfer Test: Invention F

| | |
|---|---|
| Water | Oil |

| Substrate | Styrofoam | Substrate | Styrofoam |
|---|---|---|---|
| 4 | 1 | 1 | 1 |
| 4 | 1 | 1 | 1 |
| 4 | 2 | 1 | 1 |
| 3 | 1 | 1 | 1 |

The results show that DC 7-4405, the MQ resin/dimethiconol crosspolymer, with titanium ethoxide provided the most water transfer resistance of the three lip products, although each of the inventive compositions were superior to the control.

The wear and comfort of each formulation of Table 11 were determined by panel testing. The wear test was performed by assessing each of four panelist's lip color upon initial application and after 2, 4, and 8 hours of wear. The panelists did not deviate from usual daily activities and, in each case, had lunch during the day. The wear was evaluated on a 0-5 scale, with 0 being least color retention and 5 being the most. The averaged results are presented in Table 15.

TABLE 15
Wear Test

| | Control D | Invention E | Invention F |
|---|---|---|---|
| Bare | 0 | 0 | 0 |
| Initial | 5 | 5 | 5 |
| 2 hr | 4 | 4.25 | 4.25 |
| 4 hr | 2.75 | 3 | 3.75 |
| 8 hr | 1.75 | 2.25 | 2.75 |

The wear test results show that the MQ resin/dimethiconol crosspolymer with titanium ethoxide provided the longest wear of the three lip products at longer time intervals after application.

Each sample was also evaluated for comfort. Each panelist was asked to evaluate the overall comfort of the lip stick on a scale of 1-3, with 1 being the most comfortable and 3 being the least comfortable. The averaged results are presented in Table 16 below.

TABLE 16
Comfort Test

| | Control D | Invention E | Invention F |
|---|---|---|---|
| Comfort | 2 | 3 | 1 |

The comfort test results show that the MQ resin/dimethiconol crosspolymer with titanium ethoxide was the most comfortable of the three lip products.

### Example 5

A durable hair treatment was prepared according to Table 10 below, with varying amounts of tantalum in the tantalum/dimethiconol film-former of the invention. The film former was combined with a shine enhancer, trimethylsiloxyphenyl dimethicone, to test whether it could impart durable shine to hair after one or more washings.

TABLE 10

| Durable treatment with | | | | | | |
|---|---|---|---|---|---|---|
| Aculon ALA50 | | | Control | ALA1 | ALA2 | ALA3 |
| Part | Component | Description | Wt % | Wt % | Wt % | Wt % |
| | 1653 | Dimethiconol | 5.00 | 5.00 | 5.00 | 5.00 |
| | | Aculon | | | | |
| | | ALA50 | 0.00 | 1.00 | 0.50 | 0.25 |
| | 219 | D5 | 60.00 | 59.00 | 59.50 | 59.75 |
| | 3210 | Alcohol SD-40 | 20.00 | 20.00 | 20.00 | 20.00 |
| | 3940 | Dimethicone | 10.00 | 10.00 | 10.00 | 10.00 |
| | | 50 CST | | | | |
| | 29009 | Trimethyl- | 5.00 | 5.00 | 5.00 | 5.00 |
| | | siloxyphenyl | | | | |
| | | Dimethicone | | | | |
| | | | 100.00 | 100.00 | 100.00 | 100.00 |

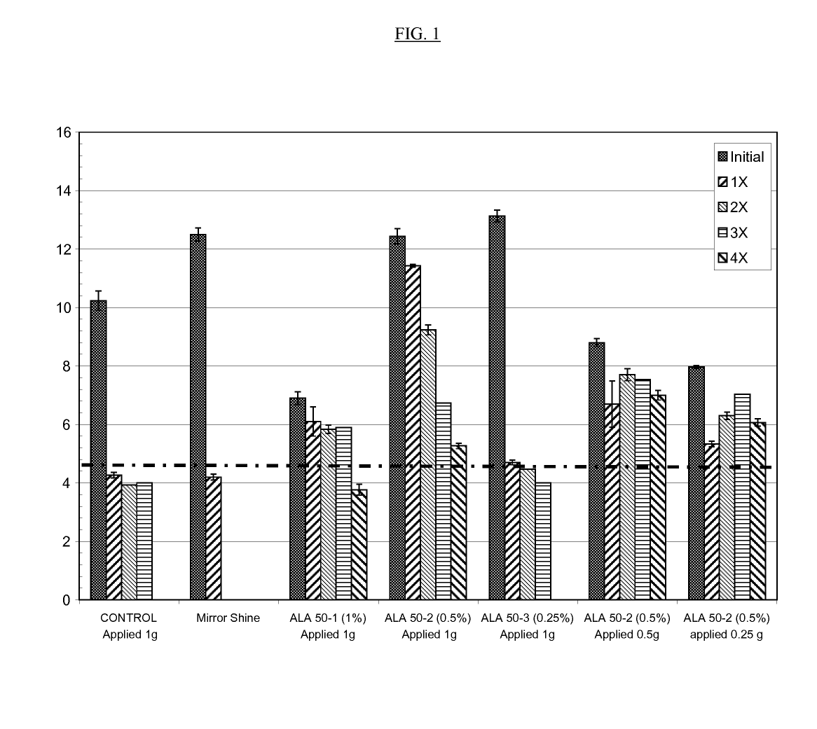

Hair was treated with the hair treatment formulations shown in Table 10, and the durability of the shine was assessed using the Bossa Nova instrument initially after application of the hair treatment, and then again after 1, 2, 3, and 4 shampooings. The purpose of the experiment was to determine if the unique formulations of the invention would impart durable shine to the hair that could last after one or more shampooings. The Bossa Nova Shine results are illustrated in FIG. 1, and demonstrate that with the control treatment, an initial shine is observed, but that shine is lost rapidly following the first shampooing. On the other hand, the inventive formulations provide a durable shine that may last throughout multiple shampooings. The best overall results were obtained with sample ALA2 when applied at a loading of 1 g to the hair tress. Even at lower applications of 0.25 g and 0.5 g, ALA2 gave durable shine over repeated washings. This phenomenon was especially apparent when comparing the decline in bossa nova shine between the first and second washings. In some embodiments, the loss of shine after one shampooing will be less than 10% or less than 20% as measured by bossa nova shine analysis. In another embodiment, the loss of shine after two shampooings will be less than about 25 or 30%.

The invention described and claimed herein is not to be limited in scope by the specific embodiments herein disclosed since these embodiments are intended as illustrations of several aspects of the invention. Any equivalent embodiments are intended to be within the scope of this invention. Indeed, various modifications of the invention in addition to those shown and described therein will become apparent to those skilled in the art from the foregoing description. Such modifications are also intended to fall within the scope of the appended claims. All publications cited herein are incorporated by reference in their entirety.

1. A film forming composition comprising the reaction product of a transition metal alkoxide with a hydroxyl-functionalized silicone polymer.

2. The film forming composition according to claim 1, wherein the transition metal alkoxide is titanium alkoxide having the form Ti(OR)₄ where R is independently selected at each occurrence from lower alkyl radicals.

3. The film forming composition according to claim 1, wherein the transition metal alkoxide is tantalum alkoxide having the form Ta(OR)₅ where R is independently selected at each occurrence from lower alkyl radicals.

4. The film forming composition according to claim 2, wherein at least one of R is ethyl.

5. The film forming composition according to claim 2, wherein at least two of R are ethyl.

6. The film forming composition according to claim 2, wherein the titanium alkoxide is titanium ethoxide having the form Ti(OCH₂CH₃)₄.

7. The film forming composition according to claim 2, wherein the tantalum alkoxide is tantalum ethoxide having the form Ta(OCH₂CH₃)₅.

8. The film forming composition according to claim 1, wherein said hydroxyl-functionalized silicone polymer is dimethiconol.

9. The film forming composition according to claim 1, wherein said hydroxyl-functionalized silicone polymer is an MQ resin.

10. The film forming composition according to claim 1, wherein said hydroxyl-functionalized silicone polymer is an MQ resin/dimethiconol crosspolymer.

11. A transfer resistant cosmetic composition comprising the film forming composition according to claim 1, and further comprising one or more of a pigment, dye, pearlescent, or lake.

12. A method of imparting a transfer resistant film to a human integument comprising depositing thereon a film forming composition according to claim 1.

13. The method of claim 12, wherein said film forming composition comprises the reaction product of titanium ethoxide and MQ resin or MQ resin/dimethiconol crosspolymer.

14. The method of claim 12, wherein said film forming composition comprises the reaction product of tantalum ethoxide and MQ resin or MQ resin/dimethiconol crosspolymer.

15. The method of claim 12, wherein said human integument is selected from hair and lips.

16. The film forming composition according to claim 3, wherein at least one of R is ethyl.

17. The film forming composition according to claim 3, wherein at least two of R are ethyl.
